(19) United States
(12) Patent Application Publication
(10) Pub. No.: US 2023/0210732 A1
(43) Pub. Date: Jul. 6, 2023

(54) REDUCTIVE TEETH WHITENING UTILIZING SALTS OF DITHIONITE

(71) Applicant: CAO Group, Inc., West Jordan, UT (US)

(72) Inventor: Steven D. Jensen, South Jordan, UT (US)

(73) Assignee: CAO Group, Inc., West Jordan, UT (US)

(21) Appl. No.: 17/568,694

(22) Filed: Jan. 4, 2022

(51) Int. Cl. A61K 8/23; A61Q 11/00

(52) U.S. Cl. CPC A61K 8/23; A61Q 11/00; A61K 2800/92

(57) ABSTRACT

A reductive composition, suitable for the dental industry, which utilizes a salt of dithionite may be provided to create an alkaline tooth whitening compound. Either Sodium or potassium dithionite may be used with marked effectiveness. A simple buffered solution of dithionite and water may whiten teeth in an alkaline environment that lessens damage to tooth ename.

### FIELD OF THE INVENTION

The present invention relates to the field of dentistry and more particularly relates to the use of reductive agents to create alkaline tooth whitening compositions.

### BACKGROUND OF THE INVENTION

Traditional tooth whitening compounds utilize oxidation reactions to whiten teeth, usually in the form of introducing peroxides to the enamel surface. These peroxide-based teeth whitening systems whiten by oxidizing the extrinsic and intrinsic stains found in teeth by removing electrons from the chromophores of stains, rendering them white. It should be noted that oxygen is not necessary for this process, however oxygen is notorious for initiating it, thus the element provides the name for the process. Hydrogen peroxide and its derivatives are stable in acidic pH environments and become increasingly unstable as the pH nears neutrality (pH 7). Bulk hydrogen peroxide is usually stored at a pH below 2. Acidic teeth whitening systems are detrimental to tooth enamel as they etch and dissolve tooth structure. Therefore, it is imperative that peroxide based whitening systems have at least a neutral pH. This is one of the main shelf-life instability factors with respect to peroxide-based tooth whiteners and the reason these compositions degrade over a short period of time. However, these whitening compounds also oxidize tooth enamel, resulting in tooth etching. As such, oxidative whitening of teeth is significantly regulated outside the United States and Canada.

Reduction reactions could also provide whitening effects. Reduction is the opposite reaction of oxidation in that electrons are added to a molecule and is often seen in conjunction with oxidation reactions (together being called redox reactions). Reduction of the chromophores of intrinsic and extrinsic enamel stains also renders them white. However, powerful reducing agents present an interesting challenge with respect to reactivity and compatibility when formulating various compositions. What usually occurs is an immediate reaction that results in a permanently reduced ingredient. For example, sodium dithionite (also known as sodium hydrosulfite, shown in the FIGURE) is a powerful reducing agent commonly used in dying processes, water treatment, gas purification, photography, and other industries. It has not been previously used in dental whitening products. Sodium dithionite is highly reactive in the presence of water, quickly decomposing to thiosulfates and bisulfates when so exposed.

The salts of dithionite are stable in alkaline pH and become increasingly unstable as the pH drops below pH 7. Therefore, a true alkaline tooth whitening system that lacks the ability to etch tooth enamel can be manufactured utilizing the dithionite anion as an active, reductive, agent.

The present invention is a stable dental whitening composition formulated from a solution of a reduction agent to provide a consumer dental whitening product. The present invention represents a departure from the prior art in that the compositions formulated using the teachings of the present invention allow for the use of highly reductive agents in wider industries, such as dentistry.

### SUMMARY OF THE INVENTION

The salts of dithionite may be used in an alkaline tooth whitening composition that will not etch or dissolve tooth enamel. The preferred salts are sodium dithionite and potassium dithionite. Nevertheless, other dithionite salts are fully capable of being utilized though not as preferred. When placed in a solution with buffered water, the resultant composition will maintain an alkaline pH and provide effective reduction of the stains found on tooth enamel.

In view of the foregoing disadvantages inherent in the known types of dental whitening products, an improved dental whitening composition may provide a chemically reductive dental composition utilizing a salt of dithionite that whitens teeth without peroxide and with the advantage of being stable in an alkaline pH

The more notable features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions as far as they do not depart from the spirit and scope of the present invention.

### BRIEF DESCRIPTION OF THE DRAWINGS

To describe the way the above-recited and other advantages and features of the invention can be obtained, a more particular description of the invention briefly described above will be rendered by reference to specific example embodiments thereof which are illustrated in the appended drawings. Understanding that these drawings depict only typical embodiments of the invention and are therefore not to be considered as limiting of its scope, the invention will be described and explained with additional specificity and detail using the accompanying drawings.

FIG. 1 is a drawing of a sodium dithionite molecule (prior art).

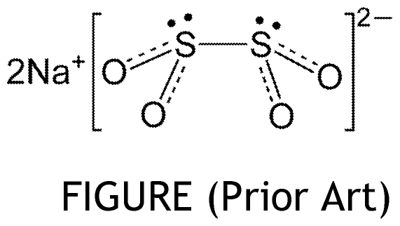

### DESCRIPTION OF THE PREFERRED EMBODIMENT

Preferred embodiments of the chemically reductive compositions are herein described. It should be noted that the articles “a,” “an,” and “the,” as used in this specification, include plural referents unless the content clearly dictates otherwise.

A reductive tooth whitening solution may be fashioned by mixing a reductive agent with water. The preferred reductive agents being the salts of dithionite and the preferred salts being sodium and potassium dithionite. Dithionite becomes most stable at a pH of or over 8.5, but the preferred pH range of the solution would be between 9.0-10.0. A buffering agent may be added to maintain the pH of the resultant solution. A pH buffer is extremely useful in maintaining a specific pH. Buffers such as sulfite, pyrosulfite, phosphate, and borax salts may be combined with a pH modifier, such as sodium or potassium hydroxide, to build a pH buffer especially above pH 8. Other pH buffering anions not listed above could also be used.

To evaluate these solutions, a collection of extracted teeth was examined and selected for dark color stains ranging from light yellow to brown. These teeth were then categorized and scored for color by comparing to a classical VITA shade guide. Then half of the teeth were then placed in a fresh solution of 15% sodium dithionite in water with unadjusted pH. The second half were placed in the solution adjusted to above pH 10. All teeth became whiter after remaining in solution overnight, especially light yellow to yellow stained teeth. The brown teeth had less of an effect but nevertheless had whitened significantly. Therefore, the results have demonstrated that dithionite does indeed whiten teeth.

Although the present invention has been described with reference to preferred embodiments, numerous modifications and variations can be made and still the result will come within the scope of the invention. The described embodiments are to be considered in all respects only as illustrative and not restrictive. No limitation with respect to the specific embodiments disclosed herein is intended or should be inferred. Therefore, the scope of the invention is indicated by the appended claims rather than by the foregoing description. All changes which come within the meaning and range of equivalency of the claims are to be embraced within their scope.

1. A tooth whitening compound comprising water, a salt of dithionite, and a phosphate buffer, wherein a final pH of the compound is greater than 9 and the phosphate creates a pH buffer greater than a pH of 8.

2. The tooth whitening compound of claim 1, the salt of dithionite being chosen from the set of salts of dithionite consisting of sodium dithionite and potassium dithionite.

3-6. (canceled)
